SEQUENCE LISTING (21 sequences)

SEQ ID NO: 1 - DNA, 19 bp, Artificial
  synthetic oligonucleotide primer with homology to human sequence
cacacggctcacattgcat

SEQ ID NO: 2 - DNA, 19 bp, Artificial
  synthetic oligonucleotide primer with homology to human sequence
cacgaacagcaaagcgacc

SEQ ID NO: 3 - DNA, 20 bp, Artificial
  synthetic oligonucleotide primer with homology to human sequence
cgtcacctcttaggcttgga

SEQ ID NO: 4 - DNA, 20 bp, Artificial
  synthetic oligonucleotide primer with homology to human sequence
cattggtgtcgttgtgctct

SEQ ID NO: 5 - DNA, 23 bp, Artificial
  synthetic oligonucleotide primer with homology to human sequence
cttcctaccctcaagtatgtccg

SEQ ID NO: 6 - DNA, 23 bp, Artificial
  synthetic oligonucleotide primer with homology to human sequence
gaggtgggaggacaggagttctt

SEQ ID NO: 7 - DNA, 23 bp, Artificial
  synthetic oligonucleotide (mir34U1) primer with homology to human sequence
nnggttttgggtaggtgtgtttt

SEQ ID NO: 8 - DNA, 23 bp, Artificial
  synthetic oligonucleotide (mir34U2.r) primer with homology to human sequence
aatcctcatccccttcaccacca

SEQ ID NO: 9 - DNA, 21 bp, Artificial
  synthetic oligonucleotide (mir34M1) primer with homology to human sequence
ggttttgggtaggcgcgtttc

SEQ ID NO: 10 - DNA, 21 bp, Artificial
  synthetic oligonucleotide (mir34M2.r) primer with homology to human sequence
tcctcatccccttcaccgccg

SEQ ID NO: 11 - DNA, 27 bp, Artificial
  synthetic oligonucleotide (mir34U3) primer with homology to human sequence
ggtttngattttttttttgtattgatg

SEQ ID NO: 12 - DNA, 23 bp, Artificial
  synthetic oligonucleotide (mir34U5.r) primer with homology to human sequence
ccaaaccccnaaaccacaacaca

SEQ ID NO: 13 - DNA, 25 bp, Artificial
  synthetic oligonucleotide (mir34M3) primer with homology to human sequence
tttngatttttttttcgtatcgacg

SEQ ID NO: 14 - DNA, 21 bp, Artificial
  synthetic oligonucleotide (mir34M5.r) primer with homology to human sequence
aaaccccnaaaccgcgacgcg

SEQ ID NO: 15 - DNA, 40 bp, Artificial
  synthetic oligonucleotide (BS1) primer with homology to human sequence
gcagacccaagaagcagtgtggaaattcactgcctgggac

SEQ ID NO: 16 - DNA, 40 bp, Artificial
  synthetic oligonucleotide (BS2) primer with homology to human sequence
tcaaagggggcatataactacatattgactgccaagaact

SEQ ID NO: 17 - DNA, 2496 bp, Homo sapiens
aatttgacctgactggccaacatggtgaaaccccgtctccacaaaaattagttgggcatagtggtgggcacctgtaatcccagctactcaggaggctgaggcaggagaatcacttgaacctgggaggtggaggttgcagtgagccagggtcaggccattgcactccagcctgggcaacaaaagtgaaactccgtctcaaaaaaaaagaaaaaagcagacctctcccttgccatgggctgcctctaggacccatctgcctccttttatccctctgagtctttgaggatctgagaagcccttcacctacttgccctgtggtgacaggcactctggactgagagtgggtatgggattgccccccagcaacaggtgtaggaaaagtaggcctgacctgcagccatccacctgcacctgggcgttctcacctgccagctggctcacggcttctctagcactgttctagaatcacggaggagtttggaaaacatgggctcatcacagacacctgtgagaataggctcaccgcccctcccctgtgagaatgggggcaggtcacccctggcctcctctcaggcccccgagggtgcccaaatgtgattacgcacctgggtagcattcgcttccctgtcctcctcctaaagaatcgtcttctcccagtgccggaggaatgagtcccccgttctcaccccggggagcggttgcctcgtgagtccaaggagaatccgccctttcgttttgcgcaggtggagatctcgcgggcccagttcggagtcctgcaaactgtgagtgtgcgaggagggcttgtttctgatgaaaccatttctgtcccgtaacaaccctgagggcagaaacccttacaaaaggagccaccgagagatttgctgtccgcttgatactggggcacggtggtcctgggggcagcttgtcccggaagggcgcggggcgcactggctgggatctataatggttggggcaggagggtgggtggggagccccttctccatggcggcgggaggtcttcggcgctgcccctgcccgcgcctccctgcccaccccaagggcgcccgaccgtgcgcgcagggcagcgtggaaagggtgggaaatgcagggagcgcgccttgggggctgccccatgctcccccctgtagctccgcgctcaggacccgcgtccgactttgcgtctctgggaacctttaaggccggtgaggggggctgagccgcggcccctcggctgagccaagccctgccccacgcaaggtccccagcctggcgcacccggctccccagcgccgcgcgccggccatgtcccttcccaccccagccccccgggacccgagagccgtgcggggccgagcctcgtgccccgaggcacgccgagccaagtcacccctgcagcccgcggcgccggaacttgggggcgcagtgagtgggggcagggcgccgggccggcgggcggtggccgtggctgggtgccggttcctggctttagaagtcctttcgcatcttgttgaatccggggctgcattggccctccgagctctccggactggcaacgtcgcagagacaacaggtcctgacccgggacagaccccacacccgcgtccaggacgtgggtttcttccctcttccaggcgagaggccctcggactgggcgtcgcggcgagccccccgtggtttctgtttgcttttgtacggtcccccttccccccaccaccctgtcccggccccacccgggccttggtcccaccgcgaaggggcgaggagtccccggaagggtcgcgatggccggggagtccgggacctcggctctgggtaggcgcgccccgcgacccagcggcggcgtgggcgaggggcgctgcgcaaactacgcgctctcgctggggaaacgccagcccaggcgggcggcggcggtgaaggggatgaggaccaggaccccgtgcccggcgggactggccccgcaccccacgccgcccccctgcggtgcgcaccgcatctgcgccctaggccgaggccaggctccgacccctcccccgcaccgacgtgattcggatcgcgcggtgctggcgccgccttcatgcgccctgcctggcccccacctggtcctctttccttttcaggtggaggagatgccgctgtcccgtcggtctggggacagcccagctccccggatcccgggctggagagacgcgtcgcggccccggggcctggtggcacgagcaggaaggaggacccggcggcgggctctgcctgggcttgcctgggcttgttccgagccgggctgcttctcggtgaccacgcagatcgggggcatttggagattttgcgggagtcctgcagccaagctccggggcaggagaggcctggaagcctgcactacctgctcgccccgtcccagcatgcacccaggtaaacgcttgtgtttctcagtccgtgcgaaagtttgcaaagaaggaggcgggaactagaccaacaactttaataatcatagtcataata

SEQ ID NO: 18 - DNA, 27 bp, Artificial
  synthetic oligonucleotide primer with homology to human sequence
gggtayggtggttttgggggtagtttg

SEQ ID NO: 19 - DNA, 30 bp, Artificial
  synthetic oligonucleotide primer with homology to human sequence
tagagataataggttttgattygggataga

SEQ ID NO: 20 - DNA, 30 bp, Artificial
  synthetic oligonucleotide primer with homology to human sequence
tctatcccraatcaaaacctattatctcta

SEQ ID NO: 21 - DNA, 31 bp, Artificial
  synthetic oligonucleotide primer with homology to human sequence
caaaactcccrcaaaatctccaaataccccc

(19) United States
(12) Patent Application Publication
(10) Pub. No.: US 2013/0096185 A1
(43) Pub. Date: Apr. 18, 2013

(54) INACTIVATION OF miR-34a BY ABERRANT CpG METHYLATION IN CANCER

(71) Applicants: Heiko HERMEKING, Munich (DE); Dmitri LODYGIN, Goettingen (DE)

(72) Inventors: Heiko HERMEKING, Munich (DE); Dmitri LODYGIN, Goettingen (DE)

(21) Appl. No.: 13/628,407

(22) Filed: Sep. 27, 2012

Related U.S. Application Data
(63) Continuation of application No. 12/490,853, filed on Jun. 24, 2009, now abandoned.
(60) Provisional application No. 61/075,152, filed on Jun. 24, 2008.

(51) Int. Cl. C12Q 1/68; A61K 45/06

(52) U.S. Cl. CPC C12Q 1/6886; A61K 45/06
USPC 514/44 R; 435/6.11; 514/49; 514/575

(57) ABSTRACT

The present application refers to methods for cancer diagnosis and treatment, particularly in types of cancers associated with aberrant CpG methylation of the miR-34a promoter.

This application is a Continuation Application of U.S. Ser. No. 12/490,853, filed Jun. 24, 2009, which claims the benefit of U.S. Serial Number 61/075,152 filed June 24, 2008, the disclosure of which are incorporated herein in their entirety by reference.

The present application refers to methods for cancer diagnosis and treatment, particularly in types of cancer associated with aberrant CpG methylation of the miR-34a promoter.

MircoRNAs (miRNAs) represent a class of endogenously expressed, small non-coding RNAs that regulate gene expression. After sequential processing steps, mature miRNAs are incorporated into the RISC complex and target specific messenger RNAs. As a consequence the translation of these mRNAs is inhibited or they are destabilized resulting in down-regulation of the encoded protein. A few miRNAs have been classified as oncogenes or tumor suppressor genes as their expression is altered in tumors and were shown to contribute to the phenotypes of cancer cells^{4,5}. Recently, miR-34a was identified as a direct target of the p53 transcription factor (reviewed in^{6,7}. Ectopic expression of miR-34a in primary and cancer cells results in apoptosis or cell cycle arrest, thereby phenocopying the effect of p53⁸⁻¹³. Among the mRNAs targeted by miR-34a are key regulators of cell cycle progression (E2F3) and apoptosis (BCL2)^{8,10,14.} These data suggest that miR-34a itself may represent a tumor suppressor gene. In fact, reduced or absent expression of miR-34a was reported in pancreatic carcinoma cell lines¹², colon cancel¹¹ and primary neuroblastoma⁸. However, a correlation between down-regulation/loss of miR-34a expression and p53 mutation or deletion of 1p36, the chromosomal region on which miR-34a resides, was not detected8,12.

It has been meanwhile established that transcriptional silencing by CpG methylation represents an important mechanism responsible for the inactivation of key tumor suppressor genes^{15,16}. Approximately, 60% of all genes harbor CpG islands in their promoter region. However, only a portion of these CpG islands display methylation of cytidine at position 5, which is accompanied by inactivation of the surrounding chromatin due to recruitment of histone deacetylases by proteins binding to methylated CpG-residues. During development this mechanism is employed to establish and maintain tissue specific expression patterns. However, during tumor formation aberrant CpG methylation occurs. Similar to genetic mutations, inactivation by CpG methylation is stably inherited on to the next cell generation and may therefore allow clonal selection of a cell population with a selective advantage during tumor progression. For example, the p16 and MLH1 genes are frequently subject to this from of inactivation in a number of different tumor types. Several other bona fide tumor suppressor genes are also inactivated by CpG methylation¹⁶. In addition, cases in which one allele of p16 or MLH1 is inactivated by CpG methylation, whereas the other allele harbors a point mutation have been described (for example in the cell line HCT116). Therefore, inactivation of tumor suppressor genes by CpG methylation may be of equal importance for the evolution of cancer cells as inactivation by genetic mutation. The exact order of molecular events that leads to aberrant silencing of tumor suppressive genes is still under debate. There is experimental evidence that histone methylation may precede DNA methylation¹⁷. Interestingly, CpG methylation seems to occur early during tumor progression and its detection may therefore be applied to tumor diagnosis and risk assessment in the future¹⁸. Since we observed that expression of miR-34a is down-regulated or lost in a number of cancer cell lines, we determined whether aberrant DNA methylation of the miR-34a promoter is a possible mode of inactivation for this microRNA in cancer.

It was found that miR-34a expression is silenced in several types of cancer due to aberrant CpG methylation of its promoter. 19 out of 24 (79.1%) primary prostate carcinomas displayed CpG methylation of the miR-34a promoter and concomitant loss of miR-34a expression. CpG methylation of the miR-34a promoter was also detected in breast (6/24; 25%), lung (7/24; 29.1%), colon (3/23; 13%), kidney (3/14; 21.4%), bladder (1/5; 20%) and pancreatic (3/19; 15.7%) carcinoma lines, as well as in melanoma cell lines (19/44; 43.2%) and primary melanoma (20/32; 62.5%). Silencing of miR-34a was dominant over its transactivation by p53/DNA damage. Re-expression of miR-34a in prostate and pancreas carcinoma cell lines induced senescence and cell cycle arrest at least in part by targeting CDK6. These results show that miR-34a represents a tumor suppressor gene which is inactivated by CpG methylation and subsequent transcriptional silencing in a broad range of tumors.

Thus, in a first aspect, the present invention refers to a method for cancer diagnosis, comprising detecting the degree of CpG methylation of the miR-34a promoter in a sample, wherein a high methylation degree is associated with cancer.

In a further aspect, the present invention refers to a method for cancer treatment in a subject suffering from a cancer associated with CpG hypermethylation of the miR-34a promoter comprising administering a therapeutically effective amount of a DNA methyltransferase inhibitor and/or a histone deacetylase inhibitor.

In a further aspect, the present invention refers to a method for cancer treatment in a subject suffering from a cancer associated with CpG hypermethylation of the miR-34a promoter, comprising administering a therapeutically effective amount of an agent which activates the p53 signal transduction pathway.

In a further aspect, the present invention refers to a method for cancer treatment in a subject suffering from a cancer associated with CpG hypermethylation of the miR-34a promotor, comprising administering a therapeutically effective amount of a chemotherapeutic agent which causes DNA damage and/or subjecting the subject to irradiation treatment.

In a further aspect, the present invention refers to a method for combined cancer diagnosis and treatment comprising
(i) detecting the degree of CpG methylation of the miR-34a promotor in a sample, wherein a high methylation degree is associated with cancer, and wherein said sample is derived from a subject suffering from cancer and
(ii) subjecting said subject to a cancer treatment assumed to be effective for the diagnosed condition.

The method of the present invention is particularly suitable for the diagnosis and/or treatment of cancer types associated with aberrant high CpG methylation of the miR-34a promoter and concomitant loss of miR-34a expression. These cancer types may be found in prostate cancer, breast cancer, lung cancer, colon cancer, kidney cancer, bladder cancer, pancreas cancer, melanoma and other cancer types. Preferably, the cancer is prostate cancer.

The present invention is suitable for cancer diagnosis and cancer treatment in medicine, particularly human medicine. Based on the diagnosis, i.e.

aberrant high CpG methylation of the miR-34a expression, suitable treatment protocols may be applied, e.g. reducing the methylation degree by administering a DNA methyltransferase inhibitor, such as 5-aza-2′-deoxycytidine, and/or a histone deacetylase inhibitor, such as trichostatin A. Alternatively, or additionally, the treatment may involve the administration of an agent which activates the p53 signal transduction pathway, e.g. by administering p53 protein as such or by administering a gene therapy vector comprising a nucleic acid molecule encoding a p53 protein. Further, this embodiment may also involve administration of miR-34a or an analogue thereof, e.g. an analogue comprising modified nucleotide building blocks, or a DNA molecule encoding miR-34a. In a further embodiment, chemotherapeutic agents may be administered which cause DNA damage such as platinum compounds. In a further embodiment, the subject may be treated with irradiation, e.g. gamma-irradiation.

Further, the present invention provides a combined cancer diagnosis and treatment protocol. In a first step, the sample derived from a subject suffering from cancer is analyzed for the degree of CpG methylation of the miR-34a promoter. Then, a treatment protocol may be applied assumed to be effective for the diagnosed condition, e.g. a treatment protocol as described above or a combination of two or more of these protocols.

In the following, the present invention is described in more detail by Figures and Examples.

### FIGURE LEGENDS

FIG. 1. Down-regulation of miR-34a expression in diverse carcinoma cell lines.

(a) RT-PCR analysis of miR-34a expression in prostate cell lines. cDNA from normal prostate epithelial cells (isolated from four donors, D1-04), four PCa and one bladder carcinoma (TsuPr1) cell line was analyzed by PCR using primers specific for pri-miR-34a and, as control, EF1α mRNA. Northern blot analysis of pri-miR-34a expression in cell lines: (b) 4 pancreatic carcinoma, (c) 2 breast carcinoma cell lines and (d) normal melanocytes (HpM61, HpM83) and 10 melanoma cell lines.

FIG. 2. Silencing of miR-34a by CpG methylation

(a) Pattern of CpG methylation in the miR-34a promoter region in prostate epithelial cells and derived cancer cell lines. The depicted region corresponds to 2.5 kbp upstream of the transcriptional start site (TSS, position indicated by arrow). Vertical bars represent CpG-dinucleotides. The position of the p53 binding site is indicated. The horizontal bars indicate amplified PCR products which were sub-cloned and analyzed by sequencing (gray: distal fragment, black: proximal fragment). The combined results obtained by sequencing of 8 independent subclones are shown for each cell line. Black areas represent the degree of cytosine methylation at the respective CpG position. The white shaded areas indicate methylation after treatment of LAPC-4 cells with 5Aza-2′dC. The open rectangles correspond to the regions covered by the MSP primers. IVM DNA was used as positive control for amplification of the M product. BPH1: benign prostate hyperplasia cells immortalized with SV40 large T antigen. IVM: in vitro methylated DNA.

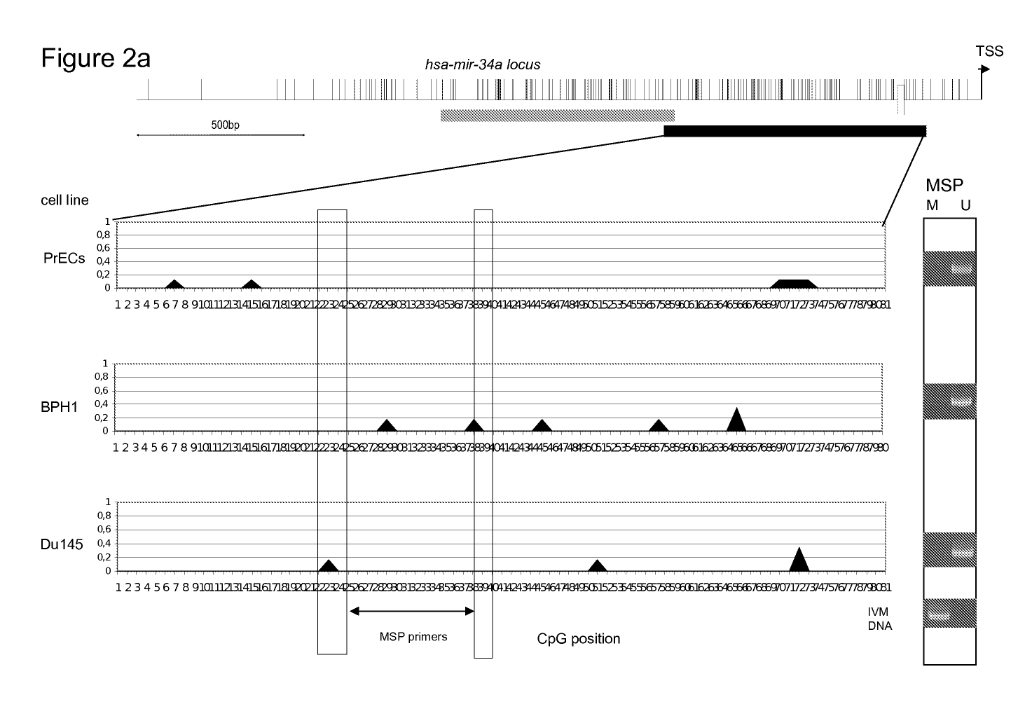

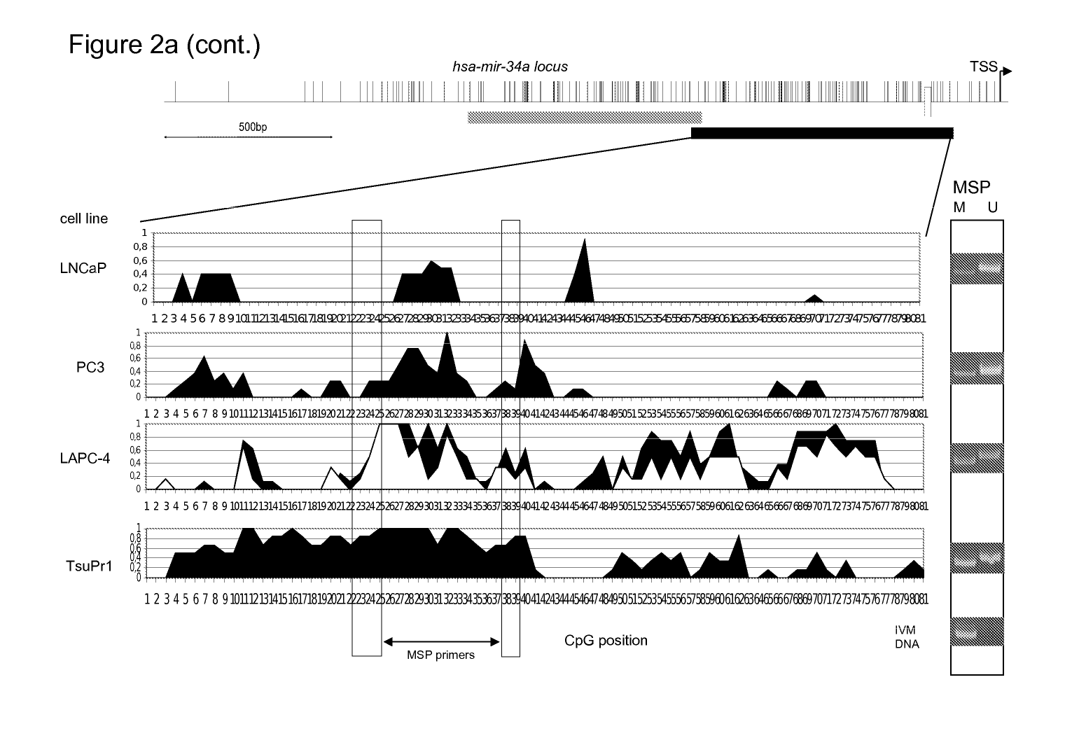

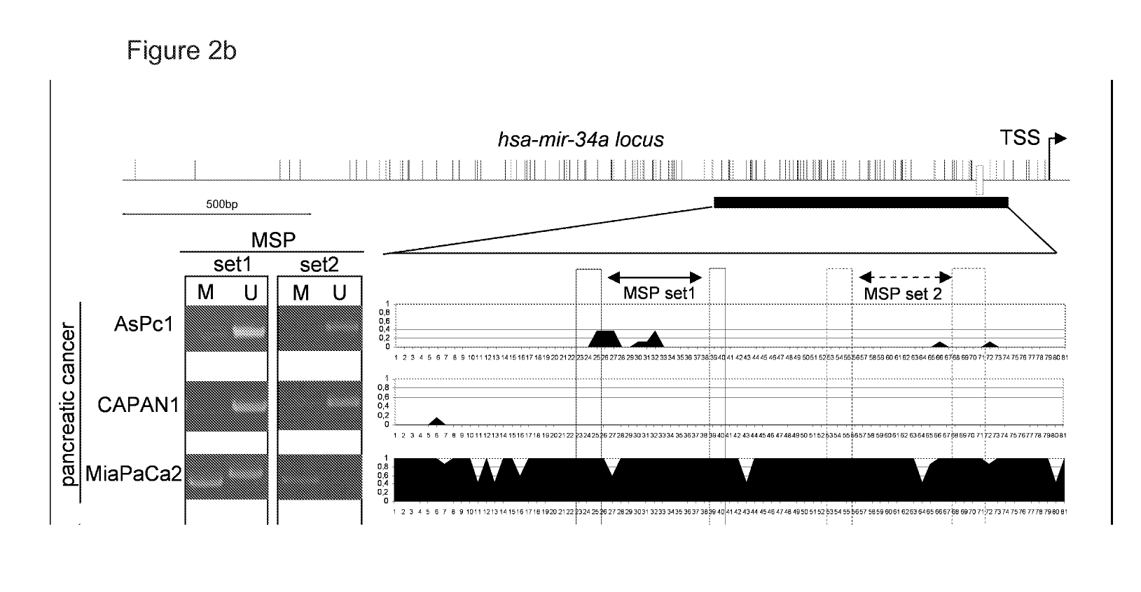

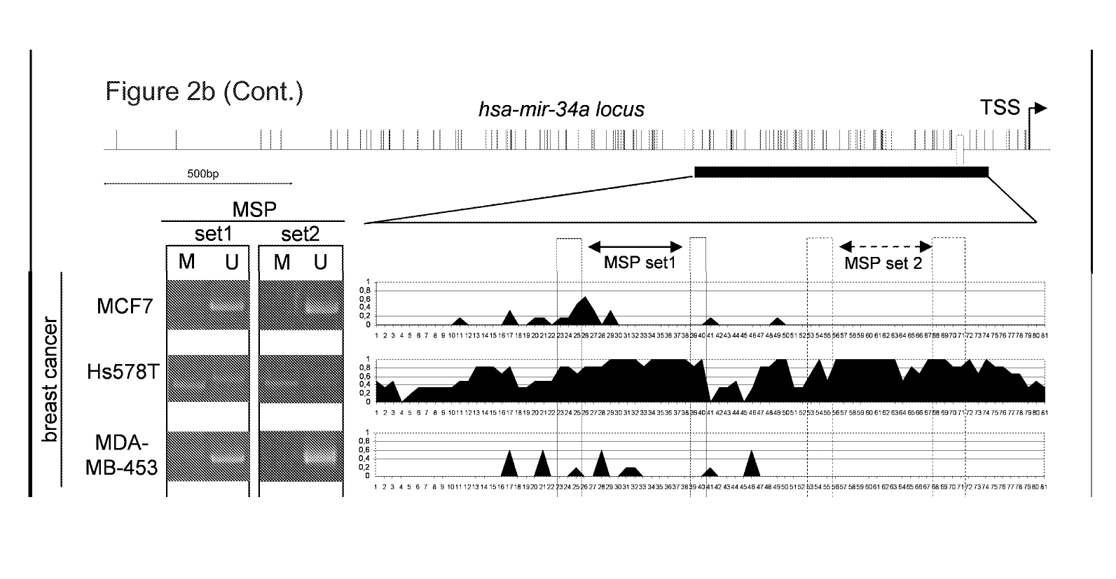

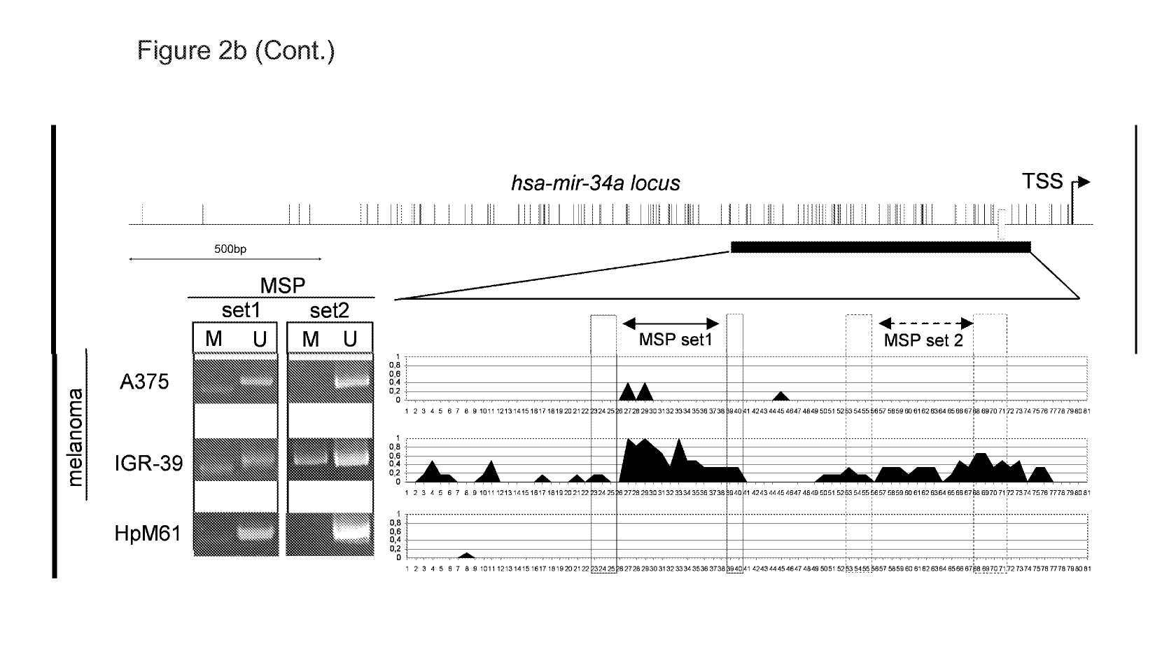

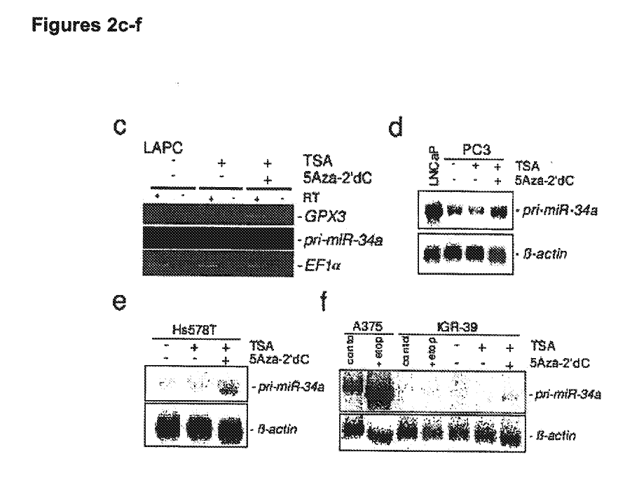

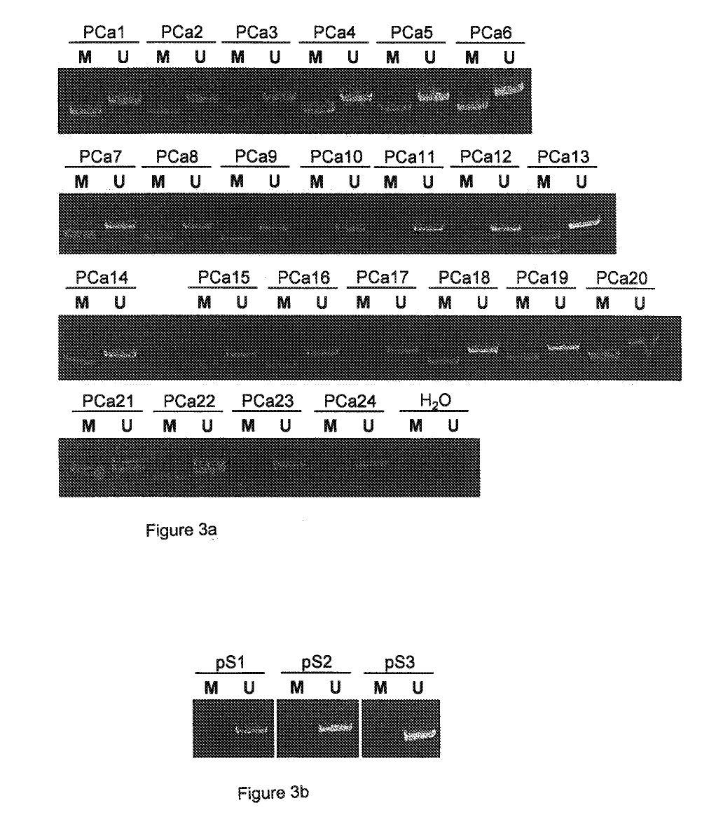

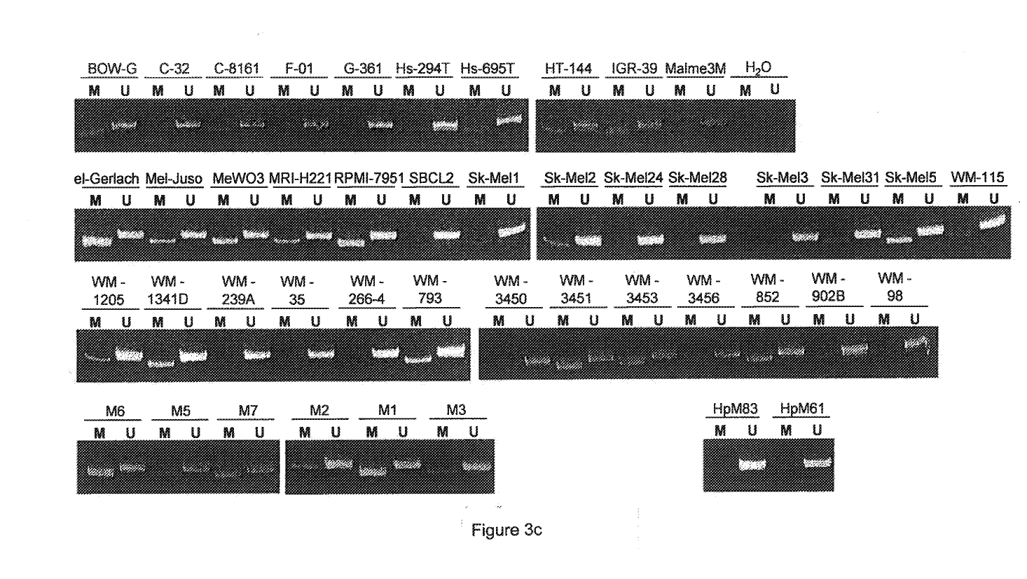

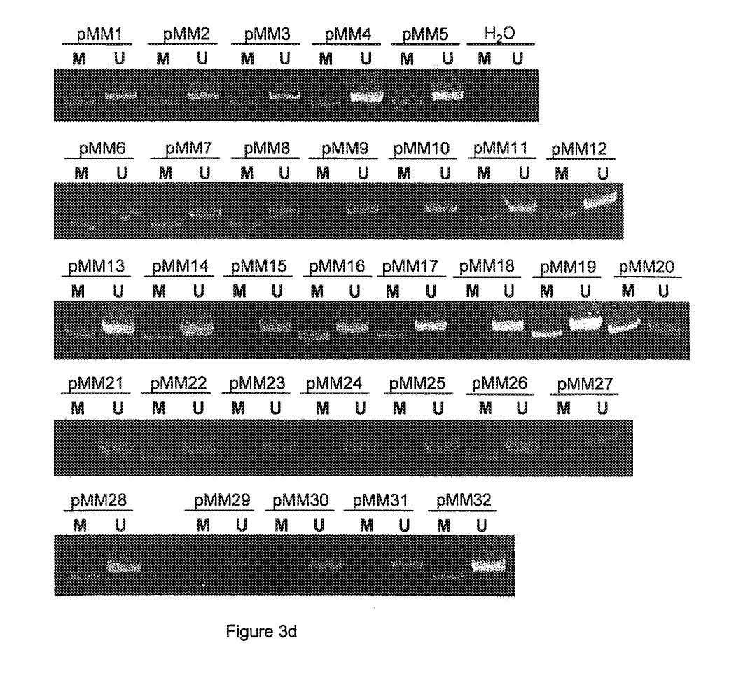

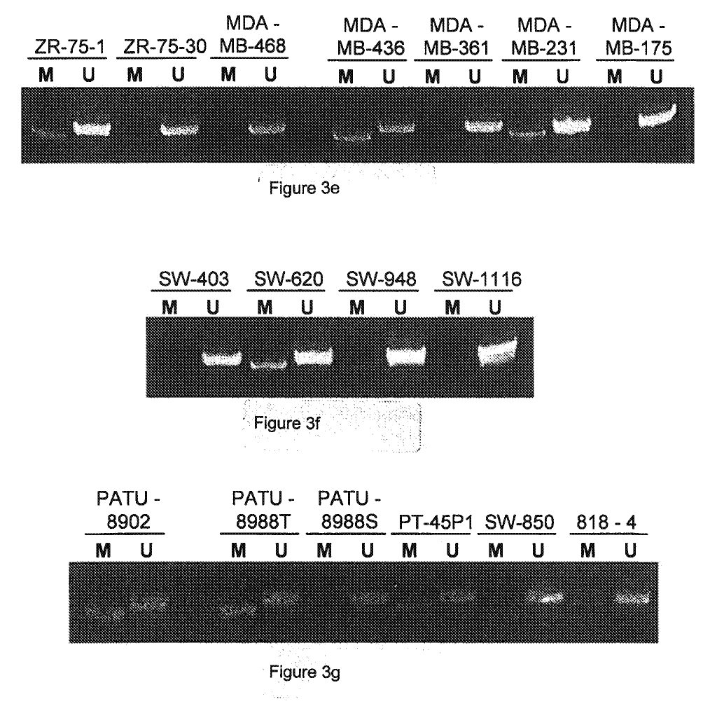

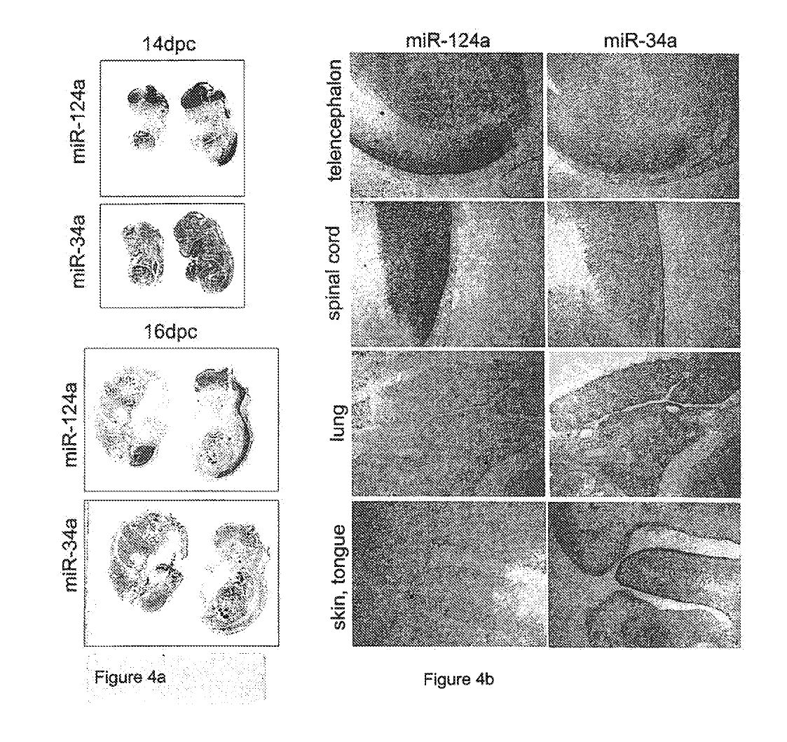

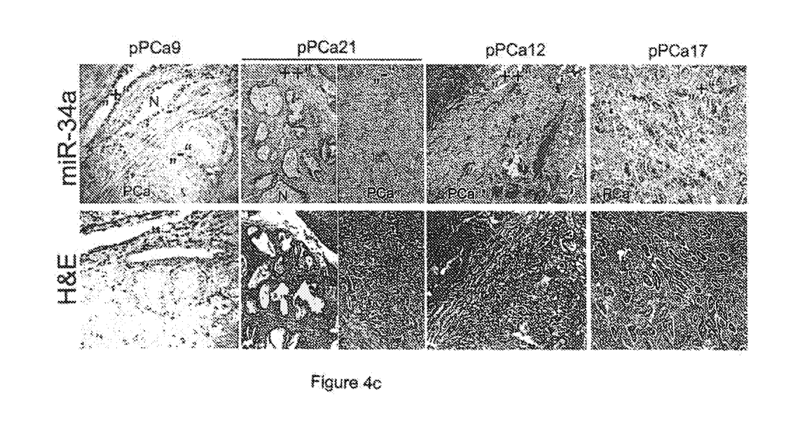

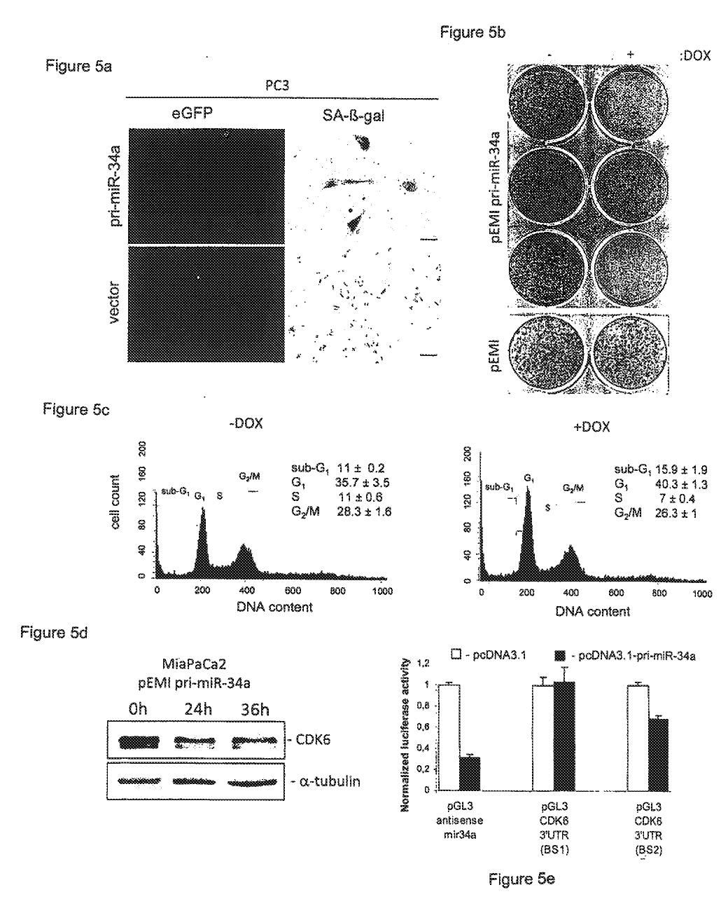

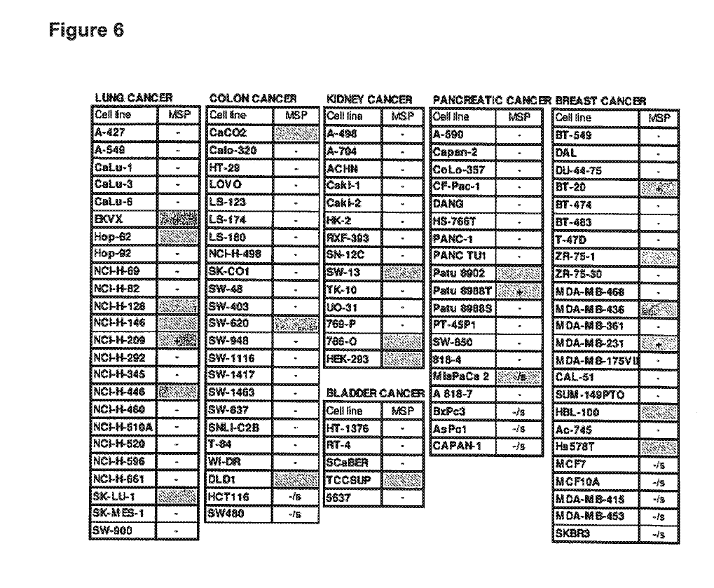

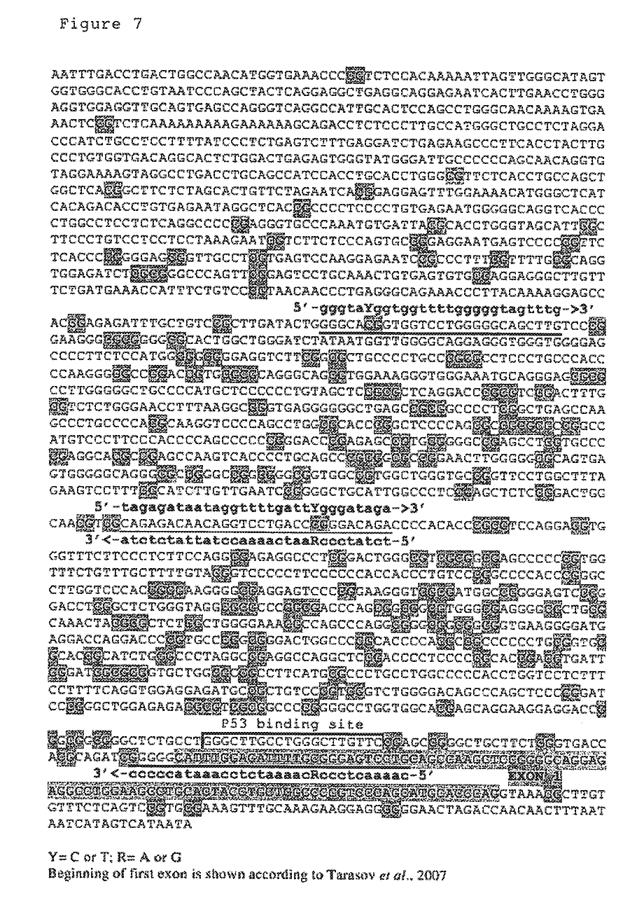

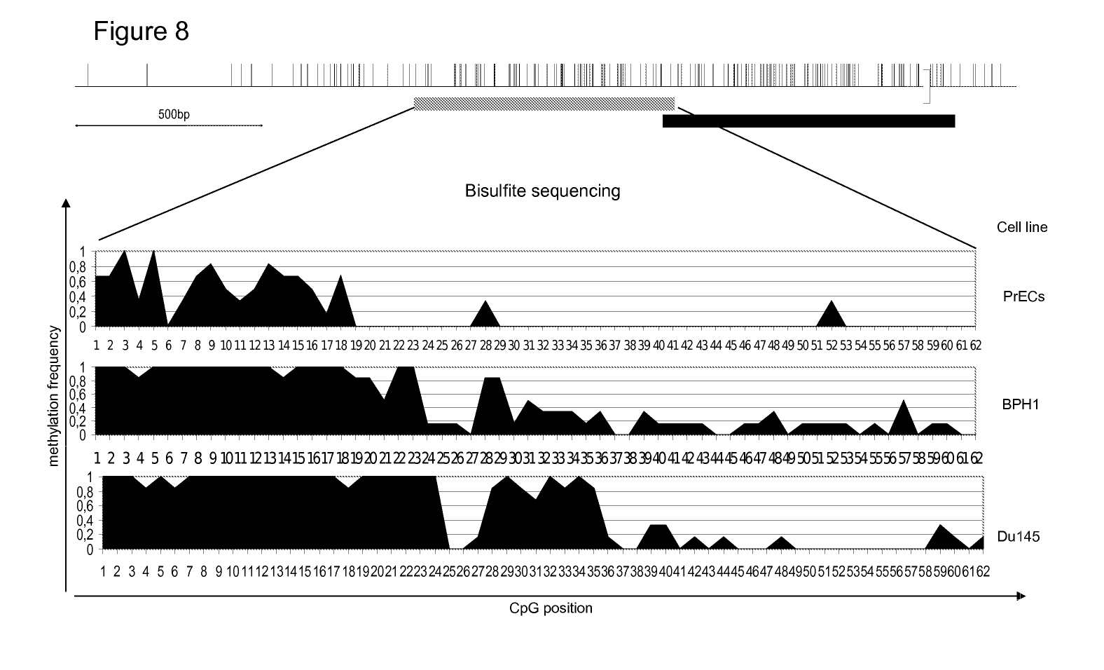

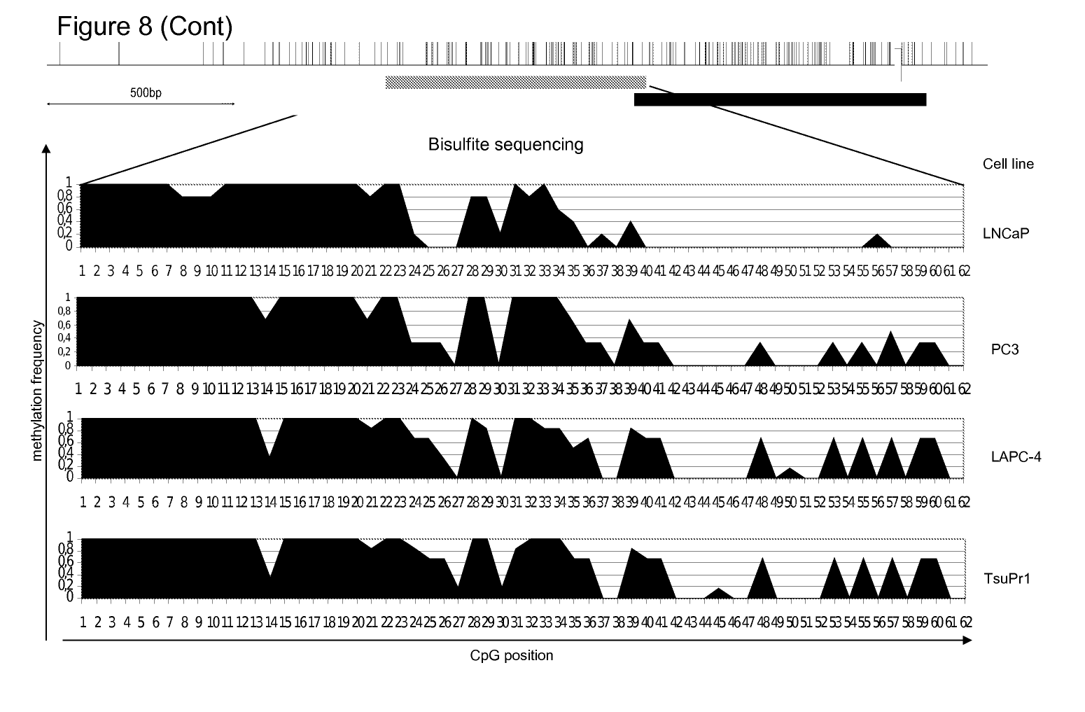

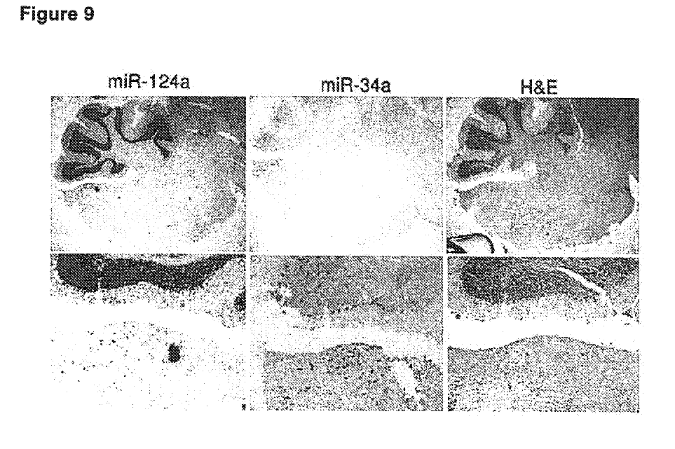

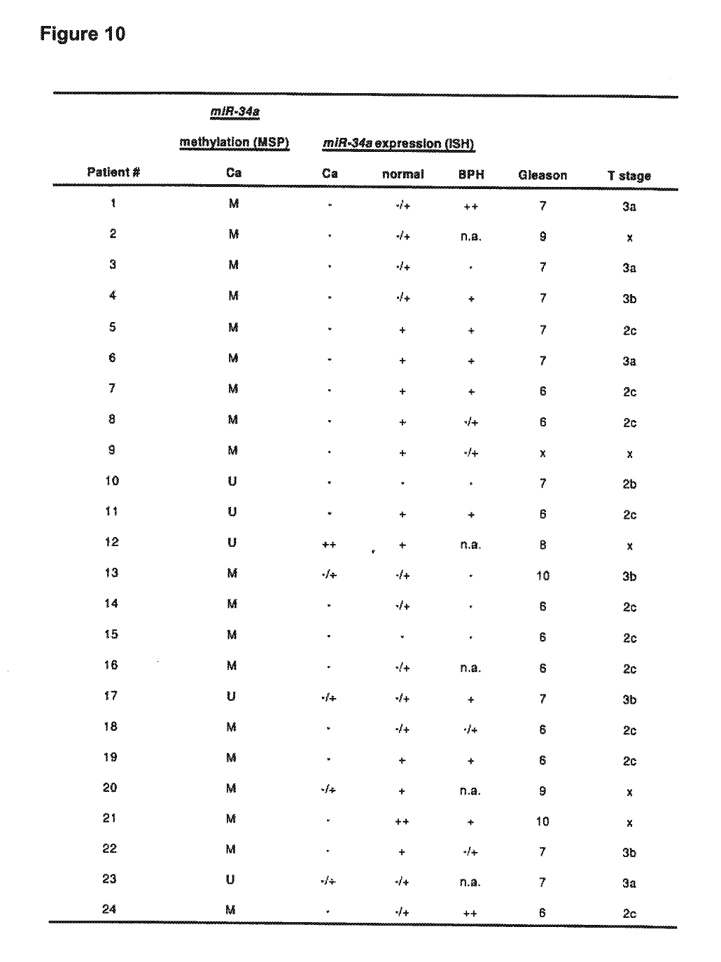

(b) MSP and bisulfit sequencing results for 3 pancreatic cancer, 3 breast cancer, and 2 melanoma cell lines. HpM61 represent human primary melanocytes. The legend of FIG. 2A applies here as well. For MSP analysis a second set of primers (MSP set 2) was developed to detect methylation in the region proximal to p53 binding site.

(c) RNA was isolated from LAPC4 cells after after treatment with TSA for 24 hours or alternatively with 5Aza-2′dC for 72 hours combined with TSA for the last 24 hours and subjected to RT-PCR with primers specific for the indicated mRNAs (for details see METHODS).

(d) Detection of pri-miR-34a expression in LNCaP and PC3 cells by Northern blot analysis. Total RNA was isolated after treatment with TSA for 24 hours or alternatively with 5Aza-2′dC for 72 hours combined with TSA for the last 24 hours. RNA was separated on a denaturing agarose gel, blotted and hybridized with radioactive probes specific for pri-miR-34a and 3-actin.

(e) Hs578T melanoma cells were treated with TSA and 5Aza-2′dC as described in Methods.

(f) For induction of DNA damage cells were treated for 24 hours with 20 μM etoposide (+etop). Hybridization with a 3-actin probe was used as loading control.

FIG. 3. Methylation specific PCR analysis of miR-34a.

DNA was isolated, subjected to bisulfite conversion and analysed by MSP with primers (set 1) specific for methylated (M) and unmethylated (U) miR-34a promoter DNA, respectively.

(a) (MSP analysis of primary prostate carcinomas (PCa). PCa cells were isolated from hematoxylin and eosin stained (H&E) formalin-fixed, paraffin-embedded prostatic sections (24 cases in total) by manual dissection of areas containing predominantely cancer cells.

(b) pS1-S3 represents material isolated from prostate sections without cancer tissue containing predominately fibromuscular stroma.

(c) MSP analysis with DNA isolated from 44 melanoma cell lines and 2 samples of primary normal melanocytes (HpM61, HpM83).

(d) MSP analysis of genomic DNA isolated from 32 samples of primary malignant melanoma tissues, and exemplary results obtained with (e) breast cancer, (f) colon cancer, and (g) pancreatic cancer cell lines.

FIG. 4. Detection of miR-34a expression by in situ hybridisation

(a) Whole embryo sections (c57Bl/6 strain) at 14 dpc (top) and 16 dpc (bottom) were hybridized with DIG-labeled LNA probes specific for mature miR-124a and miR-34a. The blue signal produced by alkaline phosphatase (AP) conjugated to anti-DIG antibodies indicates microRNA expression.

(b) Details derived from the embryo sections at 14 dpc hybridized with the indicated probes are shown. Original magnification 100x.

(c) Expression of miR-34a in primary prostate carcinoma tissues detected by LNA in situ hybridization. Exemplary results are shown. Upper images represent miR-34a expression (blue). Lower panel images depict H&E staining of the same area of a parallel tissue section. For PCa9 the original magnification is 400×; for PCa12, PCa17 and PCa21 the magnification is 100×. PCa—prostate carcinoma, N—non neoplastic prostate glands; “++”, “+” and “−” correspond to high, low or absent hybridization signals, respectively.

FIG. 5. Effects of ectopic miR-34a expression in carcinoma cells.

(a) PC3 prostate carcinoma cells were infected with a retrovirus encoding pri-miR-34a and eGFP (enhanced green fluorescent protein) on a bi-cistronic mRNA. A retroviral vector expressing eGFP served as a control. 14 days after infection cells were fixed and stained for senescence-associated ε-galactosidase at pH 6.0 (SA-β-gal). Representative examples of senescent PC3 cells (blue) are shown. The size of the bar is 100 μm.

(b) MiaPaCa2 cells transfected with the indicated constructs and selected for 14 days. Stable cell pools of were assayed for colony formation after induction of pri-miR-34a expression by addition of doxycycline (DOX). Representative wells stained with crystal violet 6 days after addition of DOX are depicted. For details see Experimental Procedures.

(c) Pri-miR-34a expression was activated by addition of DOX to MiaPaCa2 cells for 48 h. DNA content was analyzed by propidium iodide staining and flow cytometry. The percentage of cells in the respective phase of cell cycle and the standard deviation represent the analysis of three independent experiments.

(d) DOX was added for the indicated time period (hours) to MiaPaCa2 cells harboring an inducible pEMI-pri-miR-34a construct and total protein lysates were analysed by immunoblotting. Detection of □-tubulin served as a loading control.

(e) Luciferase reporter constructs containing perfectly matching target sequence of miR-34a or two putative binding sites (BS1 and BS2) for miR-34a from the CDK6 3′-UTR inserted downstream of a luciferase ORF were co-transfected with an empty vector (pcDNA3.1) or a pri-miR-34a encoding vector into H1299 cells. After 48 hours lysates were prepared and luciferase activity was measured. Results normalized for transfection efficiency derived from triplicate experiments are shown.

FIG. 6. The methylation status of miR-34a in 109 cancer cell lines.

Summary of the MSP analysis of genomic DNA isolated from 109 cancer cell lines. + and − indicate presence and absence of a distinct PCR product in the MSP reaction with methylation-specific primers (the data for MSP primers set1 are shown).

FIG. 7: Distribution of CpG dinucleotides in the miR-34a promoter region.

The primary sequence of the upstream region of human miR-34a gene is shown in capital letters. CpG-dinucleotides are marked in blue. The gray shaded area corresponds to the first exon of the miR-34a gene. The p53 binding site (BDS-1, Tarasov et al., 2007) is indicated by a rectangle. Underlined positions represent primers used for PCR amplification of templates during bisulfite sequencing analysis. The respective oligonucleotide sequences are shown in small letters.

FIG. 8:

Summary of bisulfite sequencing analysis of the distal region (gray bar) of the CpG-island in the promoter of miR-34a gene in PCa cell lines. The legend for FIG. 2A applies to this figure.

FIG. 9:

Sagittal sections of mouse brain (C57Bl/6, 2 month age) were hybridized with DIG-labeled LNA probes specific for mature miR-124a and miR-34a. A parallel section was stained with H&E. Magnification of the upper panel is 25×, lower panel 100×. MiR-124a is highly expressed in the granular layer of cerebellum and Purkinje neurons (24 h exposure to AP substrate), whereas miR-34a expression (48 h exposure) is restricted to Purkinje cells and neurons in subventricular nucleus of the medulla.

FIG. 10:

Analysis of miR-34a CpG methylation and expression in primary tissues from 24 prostate carcinoma patients.

Ca—carcinoma; normal—normal (non neoplastic) prostatic glands; BPH—benign prostate hyperplasia; n.a.—not available; x—no data; −, +, ++ denote no staining, moderate staining and pronounced staining after LNA in situ hybridization (ISH), respectively.

### EXAMPLE

### Results

miR-34a Expression is Commonly Lost in Tumor Cell Lines

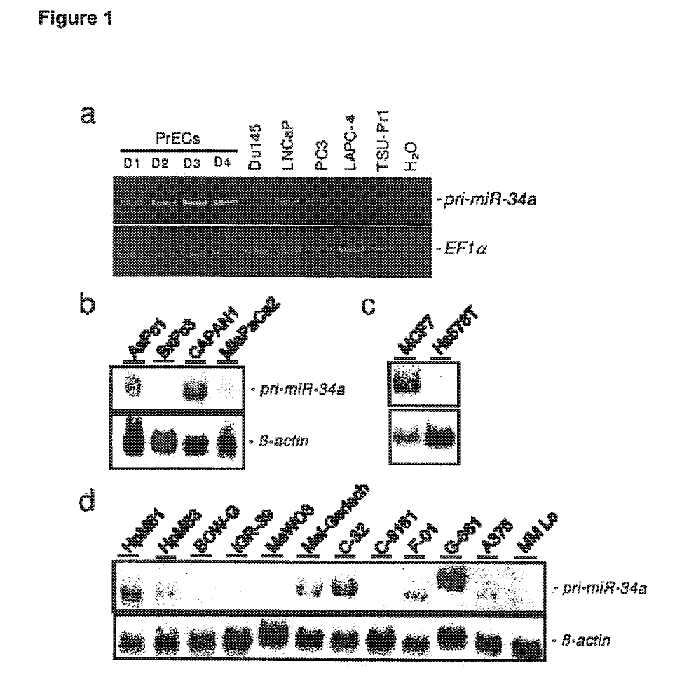

The expression of the primary miR-34a transcript (pri-miR-34a) was down-regulated or absent in the PCa cell lines Du145, PC3, LAPC-4 and the bladder cancer cell line TsuPr1, when compared to normal primary prostate cells obtained from 4 different donors or the PCa cell line LNCaP, which expresses wild type p53 (FIG. 1a). Also 2 out of 4 pancreas carcinoma, 1 of 2 breast carcinoma and 6 of 10 melanoma cell lines showed a marked down-regulation or complete loss of miR-34a expression (FIG. 1b-d). Therefore, down-regulation of miR-34a expression is a common feature of cancer cell lines derived from diverse types of cancer.

Epigenetic Silencing of miR-34a by CpG Methylation

Inspection of the genomic region upstream of the p53 binding site in the miR-34a gene revealed a prominent CpG-island (FIG. 7). We employed bisulfite sequencing to determine the CpG methylation pattern of the miR-34a promoter in prostatic cell lines (FIG. 2a). These analyses revealed that the miR-34a promoter is heavily methylated in the cell lines LAPC-4 and TsuPr1 when compared to normal PrECs or cells derived from benign prostate hyperplasia (BPH1). Therefore, the down-regulation of miR-34a observed in LAPC-4 and TsuPrl can be explained by CpG methylation. The extent of CpG methylation found in PC3 cells was moderate and in agreement with the partial down-regulation of pri-miR-34a in this cell line (FIG. 1a). The degree of CpG methylation present in the miR-34a promoter was marginal in LNCaP cells. Therefore, expression of miR-34a in LNCaP is presumably still possible from the unmethylated allele. Another reason for the more pronounced expression of miR-34a in LNCaP cells may be the presence of wild type p53. Interestingly, after treatment with 5Aza-2′dC a reduction in the level of CpG methylation of miR-34a was evident in LAPC-4 cells as determined by bisulfite-sequencing (FIG. 2a, white area). Since Du145 cells did not show CpG-methylation the down-regulation of miR-34a is presumably caused by another mechanism in this cell line. Analysis of the promoter region more distal to the transcription start site revealed uniformly dense CpG methylation in all cell lines analyzed (FIG. 8). Therefore, silencing of miR-34a expression is presumably mediated by CpG methylation of the region 100 to 500 base-pairs upstream of the miR-34a transcription start which includes the p53 binding site. In order to determine whether CpG methylation causes down-regulation of miR-34a expression, the cell lines LAPC-4 and PC3 were subjected to treatment with 5-aza-2′deoxycytidine (5Aza-2′dC) and trichostatin A (TSA), which are inhibitors of DNA methyltransferases and histone deacetylases, respectively, and synergistically reactivate the expression of genes silenced by CpG methylation. As shown previously , GPX3 was re-expressed after combined treatment with 5Aza-2′dC and TSA in LAPC-4 cells (FIG. 2c). Notably, the pri-miR34a transcript was also induced, indicating that CpG methylation is the cause for the down-regulation of miR-34a. Re-expression of pri-miR-34a after reversal of epigenetic silencing also occurred in the PCa line PC3 (FIG. 2d), but not in Du145 cells (data not shown). 5Aza-2′dC has been reported to cause DNA damage. However, since PC3 cells do not express wild-type p53, activation of p53 can be excluded as a cause for the induction of pri-miR-34a.

Pri-miR-34a was also re-expressed in the breast carcinoma cell line Hs578T and in the melanoma cell line IGR-39 after treatment with TSA and 5Aza-2′dC (FIG. 2e+f). Together with the detected presence of a highly methylated CpG-island in the promoter region of miR-34a these results convincingly show that miR-34a is subject to epigenetic silencing which critically involves CpG-methylation of promoter DNA and presumably the action of recruited HDACs. Whether, other inactivating marks are present in the chromatin of the miR-34a promoter, as methylation of residues in the histone tails remains to be shown.

Interestingly, the cell line IGR-39, which expresses wild-type p53, did not display induction of miR-34a after to exposure to etoposide, which normally serves as a strong inducer of miR-34a expression, as shown here in the melanoma cell line A375 (FIG. 2f). Therefore, CpG methylation and the accompanying inactivation of the chromatin in the miR-34a promoter, does not allow p53 to induce the expression of miR-34a mRNA. This effect may also explain how miR-34a methylation can provide a selective advantage for cancer cells, which are exposed to stimuli that lead to activation of p53.

CpG Methylation of miR-34a in Primary Tumors

To facilitate the analysis of the CpG methylation status of miR-34a in primary tumor samples we designed primers covering CpG residues specifically methylated in tumor cells (the residues methylated in LAPC-4 cells but not in PrECs, FIG. 2a). Using these primers in an MSP assay revealed unmethylated miR-34a sequences in PrECs, whereas the use of in vitro methylated DNA (IVM) as a template only gave rise to a PCR product representing the methylated allele. In general, the degree of methylation detected by MSP analysis was in agreement with results obtained by bisulfite sequencing. Similar results were obtained with a MSP primer pair positioned closer to the TSS (primer positions are indicated in FIG. 2b). Next, we analysed whether CpG methylation of miR-34a also occurs in primary tumors. MSP analysis of 24 primary prostate carcinoma tissues revealed methylation in 19 cases (79.1%), while no methylation was detected in tumor-free prostatic stroma derived from three patients (FIG. 3a+b). Therefore, CpG methylation of miR-34a is not simply a result of prolonged in vitro culture, but is already present in primary PCa at a high frequency.

CpG methylation of miR-34a is common in human cancer Next, we tested whether methylation of the miR-34a promoter also occurs in other tumor types. Therefore, we analysed genomic DNA isolated from representative cell lines derived from melanoma, breast, breast, prostate and pancreatic carcinoma using MSP (FIG. 3c, e, f, g; summarized in FIG. 6). Furthermore, primary melanoma samples were subjected to MSP analysis (FIG. 3d). The frequencies of miR-34a methylation were 7/24 (29.1%) for lung, 6/24 (25%) for breast, 3/23 (13%) for colon, 3/14 (21.4%) for kidney, 1/5 (20%) for bladder and 3/19 (15.7%) for pancreatic carcinoma cell lines. Furthermore, 19 out of 44 (43.2%) melanoma cell lines and 20 out of 32 (62.5%) primary melanomas displayed CpG methylation of miR-34a. Importantly, in primary human melanocytes the promoter of miR-34a did not display CpG methylation (HpM61 and HpM83, FIG. 3c), indicating that like in prostate epithelial cells the methylation of this CpG island is a tumor-specific event. The melanoma cell lines BOW-G, MeWO3 and M1 displayed loss of pri-miR-34a expression and also displayed cytosine methylation in miR-34a as revealed by MSP analysis (FIG. 3c), whereas G-361, C-32 and F-01 melannoma cell lines did not show CpG methylation and expressed miR-34a mRNA (FIG. 1d, 3c). For a few cell lines the loss of miR-34a expression was not associated with substantial promoter methylation (e.g. in BxPc3), indicating that alternative mechanisms of miR-34a inactivation may exist.

Detection of miR-34a Expression In Vivo

In order to determine whether CpG methylation is accompanied by decreased miR-34a expression in vivo we performed in situ hybridization (ISH) with locked nucleotide analogue (LNA) probes directed against the primary miR-34a microRNA. In mouse embryos miR-124a expression was confined to the brain and spinal cord as reported previously, while miR-34a showed a broader spectrum of positive tissues with a lower average expression level when compared to miR-124a (FIG. 4a, FIG. 9). In embryonic mouse tissues miR-34a expression was detected in several epithelial tissues (FIG. 4b), indicating that it may be subject to inactivation during the progression to carcinomas derived from these tissues.

Normal human prostatic epithelium and/or regions of benign prostatic hyperplasia (BPH) displayed robust expression of miR-34a (FIG. 4c). However, primary PCa samples displaying methylation of miR-34a did not show expression of processed miR-34a in 18 out of 19 cases (94.5%; summarized in FIG. 10). In the methylation-positive PCa13 and PCa20 carcinomas heterogeneous expression of miR-34a was observed. In these cases, miR-34a-negative areas of carcinoma showed CpG methylation. In three PCa specimens (12, 17, 23) high expression of miR-34a in carcinoma cells was accompanied by the absence of CpG methylation in miR-34a. Only in PCa11 the absence of miR-34a expression was not accompanied by promoter-methylation, suggesting that the loss of expression can also be mediated by alternative mechanisms in a few cases. In two cases (PCa10, PCa15) no ISH signals were detected in both carcinoma and non-malignant epithelial cells, which could be due to RNA degradation in these samples. In summary, the majority of primary PCa tissues displayed CpG methylation of the miR-34a promoter which was generally accompanied by loss of miR-34a expression. Therefore, miR-34a promoter methylation inversely correlates with expression of miR-34a. A correlation between the Gleason grade/pT stage and miR-34a expression was not be detected (FIG. 10).

Restoration of miR-34a Expression

Finally, we determined the effect of restoring miR-34a expression in tumor cells that had undergone loss of miR-34a expression due to epigenetic silencing. After infection of PC3 cells with a retrovirus encoding pri-miR-34a cells consistently displayed hallmarks of cellular senescence, such as enlargement, β-galactosidase activity at pH 6.0 and permanent arrest (FIG. 5a). In contrast, PC3 cells infected with a control virus displayed exponential growth (data not shown). Next, the pancreatic carcinoma cell line MiaPaCa in which miR-34a is silenced by CpG methylation was transfected with an episomal doxycycline-inducible (tet-on) expression vector driving pri-miR-34a expression or a control vector. Ectopic expression of pri-miR-34a suppressed colony formation by increasing the percentage of cells in the G₁ and sub-G₁ (apoptotic) phase, and decreasing the number of cells in S phase, whereas an empty vector had no effect on colony formation (FIG. 5b,c). As the CDK6 mRNA is a predicted target for miR-34a, we tested whether induction of pri-miR-34a leads to reduction in CDK6 protein expression in MiaPaCa2 cells. Indeed, the level of CDK6 expression was reduced at 24 and 36 hours after addition of DOX (FIG. 5d). Two putative binding sites (BS1 and BS2) present in the 3′ UTR of CDK6 mRNA were tested in a luciferase reporter assay (FIG. 5e). The miR-34a antisense sequence served as a positive control. Only the BS2-containing reporter was responsive to miR-34a. Therefore, BS2 mediates the down-regulation of CDK6 by miR-34a. These data represent an example for the modulation of a cell cycle effector by miR-34a, which may at least in part explain its anti-proliferative effect.

### Discussion

Our data show that the miR-34a gene is silenced by CpG-methylation in numerous types of cancer. This mechanism may account for the loss of miR-34a expression in a substantial portion of carcinomas, melanoma and presumably other tumor types. The highest frequency of miR-34a CpG-methylation was observed in primary prostate cancer samples, the lowest in colon cancer cell lines. It was previously shown that ectopic expression of miR-34a induces senescence in primary human fibroblasts¹⁰ and colorectal cancer cells¹¹. Our results suggest that epigenetic silencing of miR-34a in prostate cancer cells may contribute to the escape from senescence.

Detection of CpG methylation of the miR-34a gene in released tumor DNA may serve as a potential tumor diagnostic marker, as has been shown for other genes as GSTP1¹⁸. Furthermore, restoration of miR-34a function via administration of siRNAs may become a possible treatment option for cancers that show silencing of miR-34a, as siRNAs are currently being tested for cancer therapeutic purposes²³.

Interestingly, inactivation of DICER, which is required for processing of microRNAs, and the resulting decrease in microRNA expression promotes cellular transformation²⁴. In addition, activation of the c-MYC oncogene leads to global down-regulation of microRNA expression in tumor cells²⁵. Furthermore, other microRNAs (miR-127 and miR-124a) were shown to be subject to inactivation by DNA methylation^{26,27}. Taken together, microRNAs are therefore likely to have tumor suppressive functions. The high frequency of miR-34a silencing in tumors described here, suggests that miR-34a represents a tumor suppressor gene. The integration of miR-34a in the p53 network implies that its epigenetic silencing may represent a new mechanism by which tumor cells inactivate or weaken the checkpoints that involve p53.

Methods

Cell lines and tissue samples. The cell lines Du145, LNCaP, PC3, LAPC-4 and TsuPr1, BPH1 and primary human prostate epithelial cells were cultured as described previously¹⁹. Melanoma cell lines and primary human melanocytes isolated from neonatal skin were cultured as described^{28,29}. Breast cancer, bladder carcinoma, lung cancer, pancreatic cancer cell lines were cultured following recommendations of the American Type Culture Collection (ATCC) or the German Collection of Microorganisms and Cell Cultures (DSMZ, Braunschweig, Germany). Archival formalin fixed, paraff in-embedded samples of primary prostate carcinoma (Gleason sum 5-10) and cancer free prostatic tissue samples were obtained from the Institute of Pathology, Ludwig-Maximilians University, Munich and represent consecutive cases of the year 2001. All patients had undergone surgery at the same institution. In all cases two board-certified pathologists agreed on the diagnosis of PCa. Samples of primary melanoma tissue were described previously²⁹.

Reversion of genomic methylation. Cells were grown in the presence of 1 μM 5-aza-2′deoxycytidine (5Aza-2′dC, Sigma) for 72 hours and 300 nM trichostatin A (TSA, Sigma) for the last 24 hours. As controls, cells were treated with TSA only or left untreated.

Northern blot analysis. Total RNA from cell lines was isolated using RNAgents (Promega). 15 μg of total RNA were loaded per lane of denaturing 1.2% agarose gel. Detection of the primary miR-34a transcript by Northern blot hybridization was performed as described9.

RT-PCR. cDNA was generated from 1 μg total RNA per sample using anchored oligo-dT primers (Reverse-iT First Strand Synthesis; ABgene) in a total volume of 20 μl and then diluted two fold. Two μl of cDNA mixture were used for PCR with 2.5 units of Platinum Taq polymerase (Invitrogen). After 2 minutes incubation at 94° C., n PCR cycles (n=20 for EF1α, n=35 for pri-miR34a, n=36 for GPX3) were performed with 20 seconds at 94° C., 30 seconds at 60° C., 30 seconds at 72° C. and a final step 2 minutes at 72° C. The oligonucleotide sequences were: EF1α (192 by product size): 5′-CACACGGCTCACATTGCAT-3′ [SEQ ID NO: 1], 5′-CACGAACAGCAAAGCGACC-3′ [SEQ ID NO: 2]; pri-miR-34a (128 bp): 5′-CGTCACCTCTTAGGCTTGGA-3′ [SEQ ID NO: 3], 5′-CATTGGTGTCGTTGTGCTCT-3′ [SEQ ID NO: 4]; GPX3 (134 bp): 5′-CTTCCTACCCTCAAGTATGTCCG-3′ [SEQ ID NO: 5], 5′-GAGGTGGGAGGACAGGAGTTCTT-3′ [SEQ ID NO: 6]. Real-time quantitative PCR for the determination of pri-miR-34a and 3-actin expression was performed as described previously⁹.

Isolation and bisulfite treatment of genomic DNA. Genomic DNA was isolated by overnight incubation in 100 μg/ml proteinase K (Sigma) and 0.1% SDS (Sigma) at 55° C. with subsequent phenol/chloroform extraction and isopropanol precipitation. 1 μg of herring sperm DNA (Promega) was added as a carrier to DNA obtained from dissected tissue. 2 μg DNA was treated with sodium bisulfite using an EZ DNA methylation kit (Zymo Research). For the final elution from the column 16 μl elution buffer were used. 4 and 2 μl of eluate were used for bisulfite PCR and MSP reactions, respectively.

Bisulfite sequencing. Genomic DNA was isolated and treated with sodium bisulfite, which converts unmethylated cytosines into uracil residues, whereas methylated cytosines remain unchanged. In order to detect these changes two overlapping fragments covering a large portion of the CpG-island present in the promoter region of the miR-34a gene were sequenced. Bisulfite treated genomic DNA was used as a template to amplify fragments of 776 by (proximal region encompassing the transcription start site) and 690 by (distal region) with a high CpG-content (FIG. 7). After 2 minutes incubation at 94° C., 38 PCR-cycles were performed with 20 seconds at 94° C., 30 seconds at 65° C., 60 seconds at 72° C. Gel-purified PCR products were sub-cloned in a TOPO-TA vector (Invitrogen). For each cell line at least 8 individual clones were sequenced on both strands using M13 primers and BigDye terminator, and analyzed on a 3700 capillary sequencer (Applera).

MSP analysis. MSP was performed in a total volume of 20 μl using 1.5 units Platinum Taq-polymerase (Invitrogen) per reaction. Oligonucleotide sequences used for the MSP was as following: MSP set1-mir34U1 5′-IIGGTTTTGGGTAGGTGTGTTTT-3′ [SEQ ID NO: 7]; mir34U2.r 5′-AATCCTCATCCCCTTCACCACCA -3′ [SEQ ID NO: 8]; mir34M1 5′-GGTTTTGGGTAGGCGCGTTTC-3′ [SEQ ID NO: 9]; mir34M2.r 5 TCCTCATCCCCTTCACCGCCG -3′ [SEQ ID NO: 10]; MSP set2-mir34U3 5′-GGTTTIGATTTTTTTTTTGTATTGATG -3′ [SEQ ID NO: 11]; mir34U5.r 5′-CCAAACCCCIAAACCACAACACA -3′ [SEQ ID NO: 12]; mir34M3 5′-TTTIGATTTTTTTTTCGTATCGACG-3′ [SEQ ID NO: 13]; mir34M5.r 5′-AAACCCCIAAACCGCGACGCG-3′ [SEQ ID NO: 14] (where I denotes inosine). For the amplification of genomic DNA obtained from cell lines 37 and for the DNA from primary tissues 42 PCR cycles were performed. Amplified fragments were separated by electrophoresis on 8% poly-acrylamide gels and visualized by staining with ethidium bromide.

LNA in situ hybridization. A 3′-DIG labelled miRCURY™ probe specific for mature miR-34a was purchased from Exiqon (Denmark). Preparation of formalin-fixed paraffin embedded (FFPE) prostate carcinoma sections, the hybridization and detection of signals were performed according to the protocol for FFPE sections from Exiqon. The hybridization was performed with 25 nM concentration of the probe at 45° C. overnight. Anti-DIG Fab fragments conjugated with alkaline phosphatase (Roche) were used at a dilution of 1 to 1000. Sections were exposed to NBT/BCIP substrate (ready-to-use solution; Sigma) for 6 days with daily exchange of the substrate solution. When the miRCURY probe was not added to the hybridization mix no signals were obtained.

Ectopic expression of pri-miR-34a. The pri-miR34a cDNA was inserted into a retroviral vector, which also contained an eGFP expression cassette. The PCa cell line PC3 and PrECs (passage 2) were infected with empty control virus, or miR-34a expressing virus. 24 hours after infection, G418 (Sigma) was added at concentration 0.8 mg/ml for 10 days (PC3) or 0.1 mg/ml for 6 days (PrECs). Resistant cells were analyzed by fluorescent microscopy and by β-galactosidase staining at pH 6.0. To generate cell line with inducible expression of pri-miR-34a, the episomal pEMI vector with bidirectional promoter linked to mRFP cassette was used⁹. MiaPaCa2 cells were transfected with pEMI-pri-miR-34a or empty pEMI plasmid using FuGENE 6 reagent (Roche) and selected in the presence of 150 μg/mL hygromycin B for two weeks. Homogenous expression of transgene in resistant pools was verified by mRFP induction after addition of doxycycline. For colony formation assay, cells were seeded at low density in 6-well plates (2,000 cells per well) and on the next day doxycycline was added (100 ng/ml). After 6 days, colonies were fixed in 70% ethanol and stained with crystal violet. For FACS analysis, cells were treated with doxycycline for 48 hours, trypsinized and collected by centrifugation. After 2 hours fixation in 70% ethanol, cells were stained with propidium iodide and analyzed by flow cytometry (Becton Dickinson).

Western blot analysis. Cells lysates were prepared in RIPA buffer, sonicated and cleared by centrifugation. 40 mg of protein lysate per lane were separated using 12% SDS-acrylamide gels, and transferred onto Immobilon PVDF membranes (Millipore). For immunodetection, membranes were incubated with antibodies directed against CDK6 (Ab-3, K6.90, Lab Vision) or α-tubulin (DM 1A, Sigma). Signals from HRP (horse-radish-peroxidase) coupled secondary antibodies were generated by enhanced chemiluminescence solution (Millipore) and recorded with a CCD camera (440CF imaging system, Eastman Kodak Co).

Luciferase reporter assay. Oligonucleotides encompassing the hsa-miR-34a binding sites predicted by PICTAR in the 3′-UTR of CDK6 mRNA were inserted into the 3′-UTR of the luciferase gene of a modified pGL3plasmid (BS1, 5′-GCAGACCCAAGAAGCAGTGTGGAAATTCACTGCCTGGGAC-3′ [SEQ ID NO: 15]; BS2, 5′-TCAAAGGGGGCATATAACTACATATTGACTGCCAAGAACT-3′ [SEQ ID NO: 16])⁸. The control plasmid contained the perfect complementary sequence of miR-34a and was kindly provided by R. Stallings. The resulting plasmids (100 ng per well of 12 well plate) were co-transfected in H1299 cells together with either a pcDNA3.1 pri-mir-34a vector or an empty pcDNA3.1 vector. 48 h after transfection luciferase activity was determined in a dual luciferase reporter assay (Promega).

### REFERENCES

1. Filipowicz, W., Bhattacharyya, S. N. & Sonenberg, N. Mechanisms of post-transcriptional regulation by microRNAs: are the answers in sight? Nat Rev Genet 9, 102-14 (2008).
2. Meister, G. & Tuschl, T. Mechanisms of gene silencing by double-stranded RNA. Nature 431, 343-9 (2004).
3. Peters, L. & Meister, G. Argonaute proteins: mediators of RNA silencing. Mol Cell 26, 611-23 (2007).
4. Ma, L. & Weinberg, R. A. MicroRNAs in malignant progression. Cell Cycle 7(2007).
5. Calin, G. A. & Croce, C. M. MicroRNA signatures in human cancers. Nat Rev Cancer 6, 857-66 (2006).
6. Hermeking, H. p53 enters the microRNA world. Cancer Cell 12, 414-8 (2007).
7. He, X., He, L. & Hannon, G. J. The guardian's little helper: microRNAs in the p53 tumor suppressor network. Cancer Res 67, 11099-101 (2007).
8. Welch, C., Chen, Y. & Stallings, R. L. MicroRNA-34a functions as a potential tumor suppressor by inducing apoptosis in neuroblastoma cells. Oncogene 26, 5017-22 (2007).
9. Tarasov, V. et al. Differential regulation of microRNAs by p53 revealed by massively parallel sequencing: miR-34a is a p53 target that induces apoptosis and G1-arrest. Cell Cycle 6, 1586-93 (2007).
10. 10. He, L. et al. A microRNA component of the p53 tumour suppressor network. Nature 447, 1130-4 (2007).
11. Tazawa, H., Tsuchiya, N., Izumiya, M. & Nakagama, H. Tumor-suppressive miR-34a induces senescence-like growth arrest through modulation of the E2F pathway in human colon cancer cells. Proc Natl Acad Sci USA (2007).
12. Chang, T. C. et al. Transactivation of miR-34a by p53 broadly influences gene expression and promotes apoptosis. Mol Cell 26, 745-52 (2007).
13. Raver-Shapira, N. et al. Transcriptional activation of miR-34a contributes to p53-mediated apoptosis. Mol Cell 26, 731-43 (2007).
14. Bommer, G. T. et al. p53-Mediated Activation of miRNA34 Candidate Tumor-Suppressor Genes. Curr Biol 17, 1298-307 (2007).
15. Jones, P. A. & Baylin, S. B. The fundamental role of epigenetic events in cancer. Nat Rev Genet 3, 415-28 (2002).
16. Jones, P. A. & Baylin, S. B. The epigenomics of cancer. Cell 128, 683-92 (2007).
17. Bachman, K. E. et al. Histone modifications and silencing prior to DNA methylation of a tumor suppressor gene. Cancer Cell 3, 89-95 (2003).
18. Laird, P. W. The power and the promise of DNA methylation markers. Nat Rev Cancer 3, 253-66 (2003).
19. Lodygin, D., Epanchintsev, A., Menssen, A., Diebold, J. & Hermeking, H. Functional epigenomics identifies genes frequently silenced in prostate cancer. Cancer Res 65, 4218-27 (2005).
20. Karpf, A. R., Moore, B. C., Ririe, T. O. & Jones, D. A. Activation of the p53 DNA damage response pathway after inhibition of DNA methyltransferase by 5-aza-2′-deoxycytidine. Mol Pharmacol 59, 751-7 (2001).
21. Cao, X., Pfaff, S. L. & Gage, F. H. A functional study of miR-124 in the developing neural tube. Genes Dev 21, 531-6 (2007).
22. Epanchintsev, A., Jung, P., Menssen, A. & Hermeking, H. Inducible microRNA expression by an all-in-one episomal vector system. Nucleic Acids Res 34, e119 (2006).
23. de Fougerolles, A., Vornlocher, H. P., Maraganore, J. & Lieberman, J. Interfering with disease: a progress report on siRNA-based therapeutics. Nat Rev Drug Discov 6, 443-53 (2007).
24. Kumar, M. S., Lu, J., Mercer, K. L., Golub, T. R. & Jacks, T. Impaired microRNA processing enhances cellular transformation and tumorigenesis. Nat Genet 39, 673-7 (2007).
25. Chang, T. C. et al. Widespread microRNA repression by Myc contributes to tumorigenesis. Nat Genet 40, 43-50 (2008).
26. Saito, Y. et al. Specific activation of microRNA-127 with downregulation of the proto-oncogene BCL6 by chromatin-modifying drugs in human cancer cells. Cancer Cell 9, 435-43 (2006).
27. Lujambio, A. et al. Genetic unmasking of an epigenetically silenced microRNA in human cancer cells. Cancer Res 67, 1424-9 (2007).
28. Korner, H. et al. Digital karyotyping reveals frequent inactivation of the dystrophin/DMD gene in malignant melanoma. Cell Cycle 6, 189-98 (2007).
29. Berking, C., Takemoto, R., Satyamoorthy, K., Elenitsas, R. & Herlyn, M. Basic fibroblast growth factor and ultraviolet B transform melanocytes in human skin. Am J Pathol 158, 943-53 (2001).

1. A method for cancer diagnosis, comprising detecting the degree of CpG methylation of the miR-34a promoter in a sample, wherein a high methylation degree is associated with cancer.

2. A method for cancer treatment in a subject suffering from a cancer associated with CpG hypermethylation of the miR-34a promoter comprising administering a therapeutically effective amount of a DNA methyltransferase inhibitor and/or a histone deacetylase inhibitor.

3. A method for cancer treatment in a subject suffering from a cancer associated with CpG hypermethylation of the miR-34a promoter, comprising administering a therapeutically effective amount of an agent which activates the p53 signal transduction pathway.

4. A method for cancer treatment in a subject suffering from a cancer associated with CpG hypermethylation of the miR-34a promoter, comprising administering a therapeutically effective amount of a chemotherapeutic agent which causes DNA damage and/or subjecting the subject to irradiation treatment.

5. A method for combined cancer diagnosis and treatment comprising
(i) detecting the degree of CpG methylation of the miR-34a promoter in a sample, wherein a high methylation degree is associated with cancer, and wherein said sample is derived from a subject suffering from cancer and
(ii) subjecting said subject to a cancer treatment assumed to be effective for the diagnosed condition.

6. The method of claim 1, wherein the cancer is selected from prostate cancer, breast cancer, lung cancer, colon cancer, kidney cancer, bladder cancer, pancreas cancer and melanoma.
